(19) United States
(12) Patent Application Publication
(10) Pub. No.: US 2016/0243049 A1
(43) Pub. Date: Aug. 25, 2016

(54) NANORASPBERRIES FOR PHOTOTHERMAL CANCER THERAPY

(71) Applicant: Washington University, St. Louis, MO (US)

(72) Inventors: Srikanth Singamaneni, St. Louis, MO (US); Naveen Gandra, Durham, NC (US); Christopher Portz, St. Louis, MO (US)

(21) Appl. No.: 15/048,605

(22) Filed: Feb. 19, 2016

Related U.S. Application Data
(60) Provisional application No. 62/118,164, filed on Feb. 19, 2015.

(51) Int. Cl. A61K 9/51; A61K 41/00; A61N 5/06; A61K 33/24

(52) U.S. Cl. CPC A61K 9/5161; A61K 33/24; A61K 9/5192; A61K 41/0052; A61N 5/062; A61N 5/0625; A61N 2005/0659; A61N 2005/0662; A61N 2005/067

(57) ABSTRACT

Compositions and methods for cancer therapy are disclosed. More particularly, the present disclosure relates to tumor-selective chitosan protected gold nanoraspberries for photothermal cancer therapy.

### CROSS-REFERENCE TO RELATED APPLICATIONS

This application claims the benefit to U.S. patent application Ser. No. 62/118,164, filed Feb. 19, 2015, which is incorporated herein by reference in its entirety.

### STATEMENT REGARDING FEDERALLY SPONSORED RESEARCH OR DEVELOPMENT

This invention was made with government support under Grant CBET-1254399 awarded by National Science Foundation CAREER award. The Government has certain rights in the invention.

### BACKGROUND OF THE DISCLOSURE

The present disclosure relates generally to nanoparticles for cancer therapy. More particularly, the present disclosure relates to tumor-selective chitosan protected gold nanoraspberries for photothermal cancer therapy.

Nanomedicine holds great promise in revolutionizing the way cancer is diagnosed, imaged, and treated. For homing in on the tumor site, most nanoscale drug delivery systems rely on enhanced permeation and retention (EPR) effect caused by leaky vasculature and poor lymphatic drainage of the tumor. The effectiveness of the EPR effect mainly depends on the colloidal stability and blood circulation time of nanostructures under physiological conditions, which necessitates the modification of these nanostructures with “stealth” coatings such as polyethylene glycol (PEG) brushes to delay their uptake by macrophages and prolong their blood circulation time. Although the polymer coatings enhance the serum stability and blood circulation time, they also hinder the desired nanoparticle uptake by cancer cells.

Targeted delivery of nanostructures to a tumor site often requires further modification of the nanostructures with disease recognition elements such as antibodies and aptamers. This modification requires additional steps such as production, purification, conjugation, and sterilization of the nanotherapeutics. These steps, especially at nanoscale, are very sensitive and expensive, which makes it difficult to translate most of the nanotherapeutics to clinical applications.

Owing to their unique optical properties such as large absorption and scattering cross section and large enhancement of electromagnetic field at the surface, plasmonic nanostructures have received extensive attention as a highly promising class of materials for nanooncology. Most of the existing plasmonic nanostructures require extensive post-synthesis treatments and biofunctionalization routines to mitigate their cytotoxicity and/or make them tumor-specific.

These considerations highlight the need for easy-to-synthesize, biocompatible, highly stable and cancer specific nanotherapeutics.

### BRIEF DESCRIPTION OF THE DISCLOSURE

The present disclosure relates generally to nanoparticles for cancer therapy. More particularly, the present disclosure relates to tumor-selective chitosan protected gold nanoraspberries for photothermal cancer therapy.

In one aspect, the present disclosure is directed to a composition comprising: a plurality of gold nanoparticles; and a chitosan-coating surrounding the plurality of gold nanoparticles, wherein the composition has a raspberry-like morphology.

In another aspect, the present disclosure is directed to a method of preparing a plurality of chitosan-coated gold nanoraspberries, the method comprising: forming a growth solution, wherein the growth solution is prepared by providing a chitosan solution; adding to the chitosan solution a solution comprising gold chloride (HAuCl₄); adding a solution comprising silver nitrate (AgNO₃) to the chitosan solution; adding ascorbic acid; and incubating the growth solution for a sufficient time to form the plurality of chitosan-coated gold nanoraspberries.

In another aspect, the present disclosure is directed to a method of photothermal cancer treatment in a subject having or suspected of having a cancer tumor, the method comprising: administering a plurality of chitosan-coated gold nanoraspberries to the subject; incubating the subject for a sufficient period of time to allow for internalization of the chitosan-coated gold nanoraspberries by cells of the cancer tumor; and exposing the cancer tumor to laser irradiation.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1A is a schematic representation of a chitosan protected gold nanoraspberry (GRB) and the chemical structure of chitosan.

FIG. 1B are TEM images of GRBs (scale bar is 200 nm) and a single GRB (inset; scale bar is 40 nm).

FIG. 1C-1G are TEM images of GRB synthesized with 0.5 mg chitosan (FIG. 1C); 1.25 mg chitosan (FIG. 1D); 2.5 mg chitosan (FIG. 1E); 5 mg chitosan (FIG. 1F); and 10 mg chitosan (FIG. 1G). Scale bar is 50 nm.

FIG. 1H is a TEM image of a GRB revealing an approximate 20 nm-30 nm chitosan layer.

FIG. 1I is a graph depicting Vis-NIR extinction spectra of GRB synthesized with 1.25 mg/ml chitosan; 2.5 mg/ml chitosan; 3.75 mg/ml chitosan; and 5 mg/ml chitosan.

FIG. 1J is a graph depicting thermogravimetric analysis of GRB to show percentage weight of chitosan and its transition temperature between 400° C. and 800° C.

FIG. 2A is a schematic representation of GRBs formation from a highly open 3D chitosan polymer scaffold to an intermediate stage wherein nanoparticle seeds interact with chitosan sites to final stage chitosan-coated gold nanoraspberries.

FIGS. 2B-2D are TEM images of intermediate structures at time intervals from 1 minute (FIG. 2B); 2 minutes (FIG. 2C); and 10 minutes (FIG. 2D)) at different stages of GRBs formation. Scale bar=50 nm.

FIG. 3 illustrates the pH dependent serum stability of GRBs. FIG. 3A is a graph depicting Zeta potential and hydrodynamic size of GRBs at both physiological (−7.3) and tumorigenic (6.0) pH. FIG. 3B is a graph depicting time dependent formation of protein corona on GRBs and subsequent aggregation of GRBs at pH 7.3 and 6.3.

FIGS. 3C and 3D are graphs depicting Vis-NIR extinction spectra of GRBs after incubating with 10% and 100% serum at pH 7.3 (FIG. 3C) and pH 6.5 (FIG. 3D).

FIG. 3E depicts a photographic image showing aggregation and sedimentation of GRBs at the bottom of the cuvette at pH 6.3 and remaining suspended at pH 7.3.

FIG. 3F is an image depicting the X-ray crystal structure of BSA.

FIG. 3G is a schematic representation of protein corona formation at both physiological (−7.3) and tumorigenic (−6.0) pH.

FIG. 4 is a graph depicting the cytotoxicity of GRBs.

FIG. 5A is a graph depicting FT-IR of GRBs (“Raspberries”) and FITC-GRBs (FITC-Raspberries) showing the difference in relative intensity between 1° and 2° amine after carbodimide coupling, which confirms the successful chemical conjugation.

FIG. 5B are bright field and corresponding fluorescence images of SKBR-3 and MCF-10A cells after incubation with FITC-GRBs for 6 hours showing the cancer selective uptake of GRBs (scale bar is 100 μm).

FIG. 5C is a TEM of an SKBR-3 cell revealing internalized GRBs (white arrows).

FIG. 5D is a TEM of an MCF-10A cell revealing the absence of GRBs under the same incubation conditions as the SKBR-3 cells depicted in FIG. 5C.

FIGS. 6A-6D depict bright field, dark field and fluorescent images of photothermal cancer therapy. FIGS. 6A and 6B rows depict SKBR-3 cells and FIGS. 6C and 6D rows depict MCF-10A cells incubated with 10 ng/ml of GRBs. Rows in FIGS. 6A and 6C rows correspond to images of cells not irradiated with a laser and rows in FIGS. 6B and 6D correspond to those irradiated with a laser. All unexposed cells shows only fluorescence in “Live” column, which indicates that all the untreated cells are alive (i.e., GRBs alone do not result in any toxicity). In the case of exposed cells, only SKBR-3 cells are found to be dead as indicated by fluorescence in FIG. 6B “Dead” while the MCF-10A cells are unaffected by the treatment as indicated by the green fluorescence in FIG. 6D “Live”. Columns are bright field, dark field, green fluorescence channel (live), and red fluorescence channel (dead) microscopy images, respectively.

FIGS. 7A and 7B are fluorescence micrograph images depicting selective photothermal therapy and quantification of co-cultured cells with live/dead staining after laser exposure in the absence (FIG. 7A) and presence (FIG. 7B) of GRBs.

FIG. 7C is a graph depicting flow cytometry of GRBs targeted co-cultured cells to quantify the number of live and dead cells after photothermal treatment.

FIG. 7D is a graph depicting viability of SKBR-3 and MCF-10A cells after photothermal therapy at different concentration of GRBs. After photothermal therapy, most of the SKBR-3 cells are dead even at very low concentration whereas 98% of MCF-10A cells are viable.

FIG. 8 is a graph depicting an MTT assay of SKBR-3 cell to determine the cell viability at different concentrations of GRBs.

FIGS. 9A-9D are graphs depicting the comparison of SKBR-3 cell viability in the presence of GRBs with and without Ag at pH 7.5 after incubating for 24 hours (FIG. 9A); at pH 7.5 after incubating for 48 hours (FIG. 9B); at pH 6.5 after incubating for 24 hours (FIG. 9C); and at pH 6.5 after incubating for 48 hours (FIG. 9D).

FIGS. 10A and 10B are graphs plotting the zeta potential of GRBs at pH 7.5 (FIG. 10A) and pH 6.5 (FIG. 10B).

FIG. 10C is a plot of the hydrodynamic size distribution of GRBs using dynamic light scattering at pH 7.0, pH 7.5, and pH 6.5.

FIG. 10D is a plot showing both zeta potential and size at pH 7.0, pH 7.5, and pH 6.5.

FIG. 11 is a schematic illustration depicting the chemical conjugation of fluorescein to chitosan.

FIGS. 12A and 12B are graphs depicting thermogravimetric analysis of GRBs from 200° C. to 1000° C. to show the presence of percentage weight loss of chitosan (FIG. 12A) and weight loss of chitosan (FIG. 12B). The organic content was burnt between 400° C. and 800° C. confirming the transition temperature of chitosan (FIG. 1J).

FIGS. 13A and 13B are graphs depicting the viability of SKBR-3 and MCF-10A cells in the presence of chitosan between 75 ng/ml to 375 ng/ml (FIG. 13A) and 50 μg/ml to 250 μg/ml (FIG. 13B).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

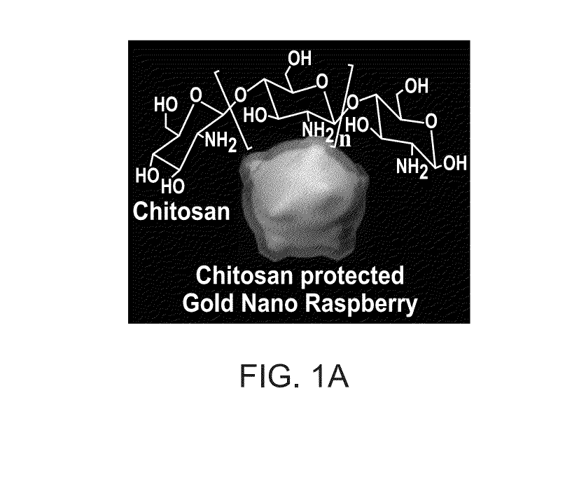

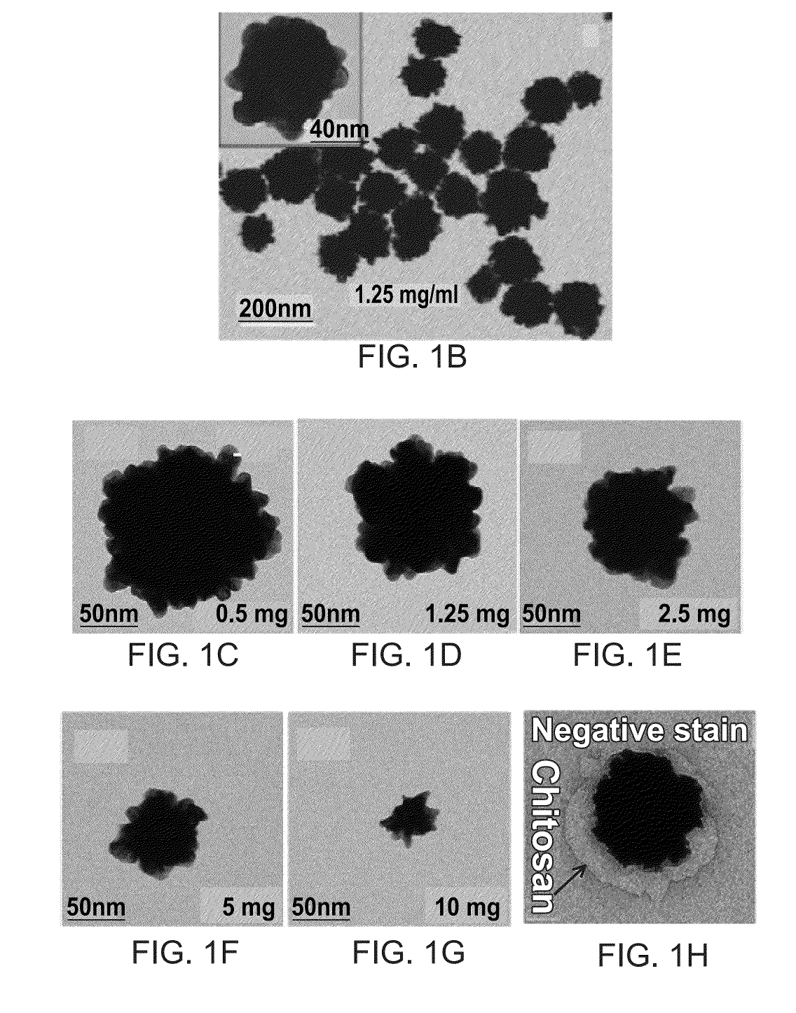

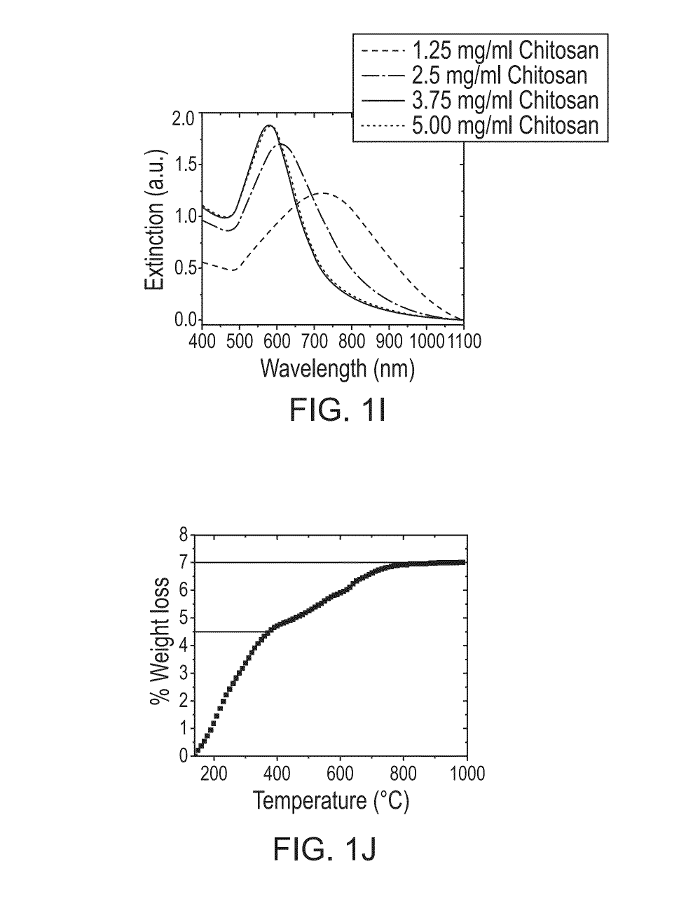

Provided herein are plasmonic nanostructures, namely, gold nanoraspberries (GRBs) with tunable size and localized surface plasmon resonance (LSPR) in the near infrared (NIR) therapeutic window (650 nm-900 nm). The gold nanoraspberries incorporate chitosan, which acts as a template and capping agent. Without be bound by theory, chitosan may also act as a biocompatible stabilizing agent, obviating the need for conventional toxic surfactants and multi-step ligand-replacement procedures (FIG. 1A). The GRBs synthesized using chitosan exhibit high (i) serum stability; (ii) biocompatibility; (iii) tunable optical properties; (iv) pH sensitivity; and (v) cancer selectivity, which are highly desirable for translation of plasmonic nanomedicine into routine medical practices.

Significantly, the GRBs, without need for any further biofunctionalization, exhibit selectivity to tumor cells, thus enabling locoregional therapy at the cellular level with minimal systemic toxicity. The tumor-selectivity of GRBs may be used with photothermal ablation to selectively ablate cancer cells while limiting damage to healthy cells. The simple, scalable, and tumor-selective nature of GRBs makes them excellent candidates for translational plasmonic nanomedicine.

Further provided herein is a synthesis method for gold nanoraspberries. The synthesis method allows for a simple and scalable process for producing the GRBs without the need for further post-synthesis treatment or biofunctionalization.

I. Nanoraspberries

In one aspect, the present disclosure is directed to a composition comprising: a plurality of gold nanoparticles; and a chitosan-coating surrounding the plurality of gold nanoparticles, wherein the composition has a raspberry-like morphology.

In various aspects, gold nanoraspberries for photothermal therapy can include chitosan as a stabilizing agent in addition to providing stealth properties to the GRBs. The strong optical absorption of GRBs in the therapeutic optical window makes GRBs excellent for photothermal therapy, while the addition of chitosan can allow the GRBs to target cancer cells without further processing or biofunctionalization with targeting agents.

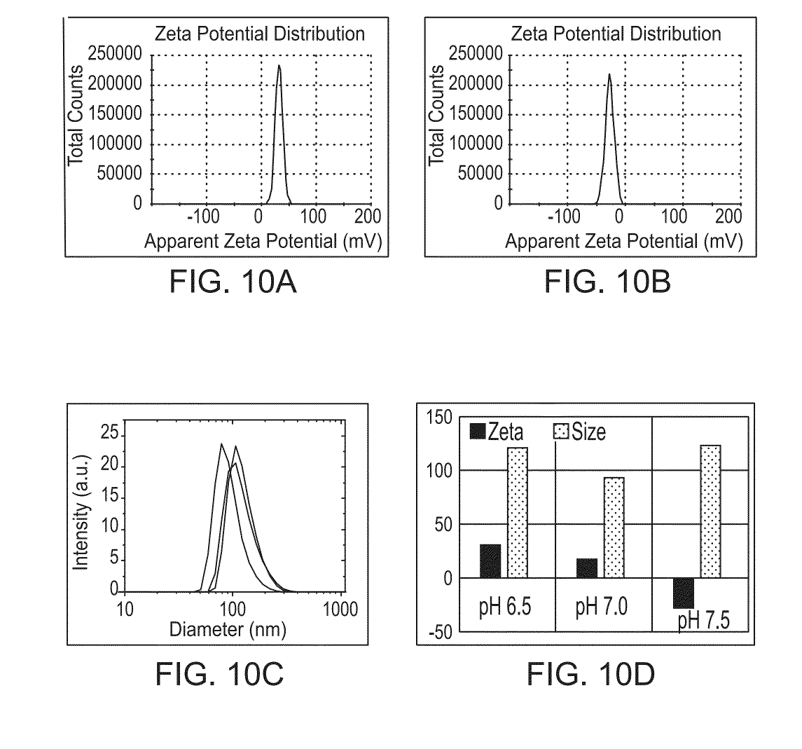

The GRBs can have a raspberry-like morphology, also referred to herein as a nanocluster or nanopopcorn-like shape, where smaller nanoparticles are clustered to form slightly larger nanoparticles. The GRBs composition has a diameter ranging from about 100 nm to about 150 nanometers. The GRBs can be monodisperse with a diameter of about 130±13 nm. In an aspect, GRBs can be about 130 nm in diameter when synthesized using 1.25 mg/ml of chitosan and have an LSPR peak at about 780 nm (FIG. 1I). In an aspect, GRBs can be synthesized using 2.5 mg/ml of chitosan and have an LSPR peak at about 625 nm (FIG. 1I). In an aspect, GRBs can be synthesized using 3.75 mg/ml of chitosan and have an LSPR peak at about 580 nm (FIG. 1I). In an aspect, GRBs can be synthesized using 5.00 mg/ml of chitosan and have an LSPR peak at about 580 nm (FIG. 1I). The thickness of the layer of chitosan on the GRBs can range from about 20 nm to about 30 nm (FIG. 1H). The thickness of the chitosan layer on GRBs obtained from TEM analysis is consistent with the hydrodynamic diameter of GRBs measured using dynamic light scattering (FIG. 10C and 10D). In an aspect, the GRBs composition includes about 1% to about 10% chitosan content as measured by thermogravimetric analysis. In an aspect, the GRBs composition includes about 90% to about 99% gold content as measured by thermogravimetric analysis (as depicted in FIGS. 12A and 12B). In an aspect, the GRBs may include about 2.5% organic (chitosan) and about 97.5% of inorganic (gold) content, which is consistent with the TEM data as seen in FIG. 1H. The GRBs composition can have a localized surface plasmon resonance peak ranging from about 650 nm to about 900 nm. The GRBs composition can further include a label. Suitable labels include, for example, fluorescent labels.

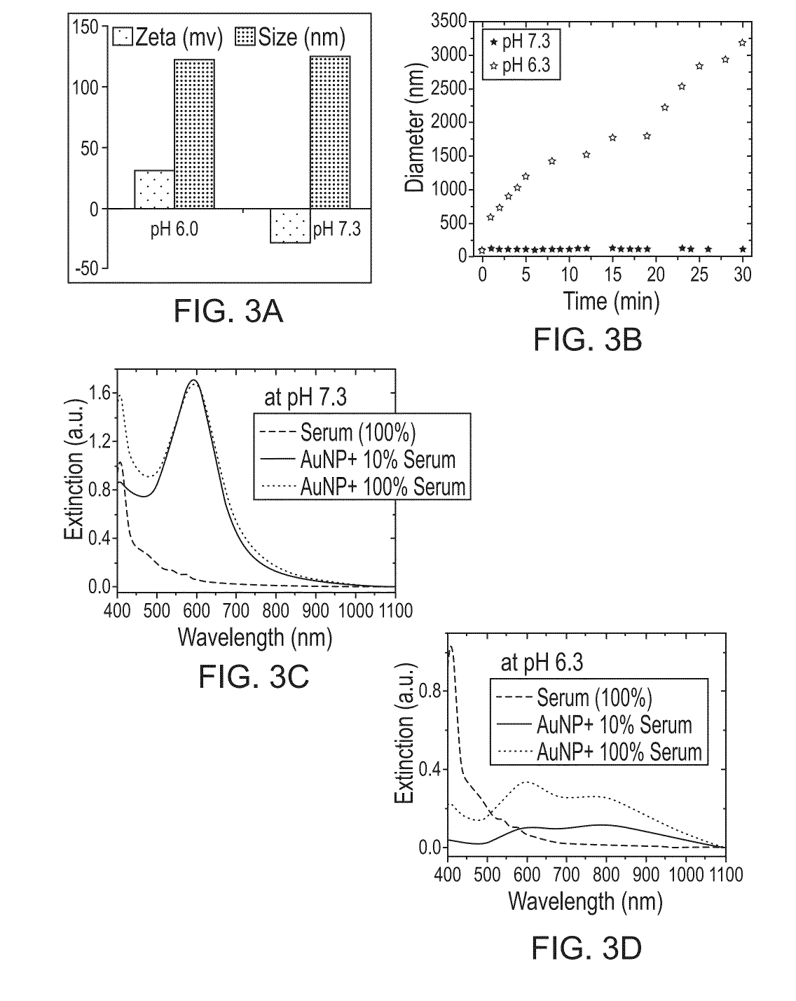

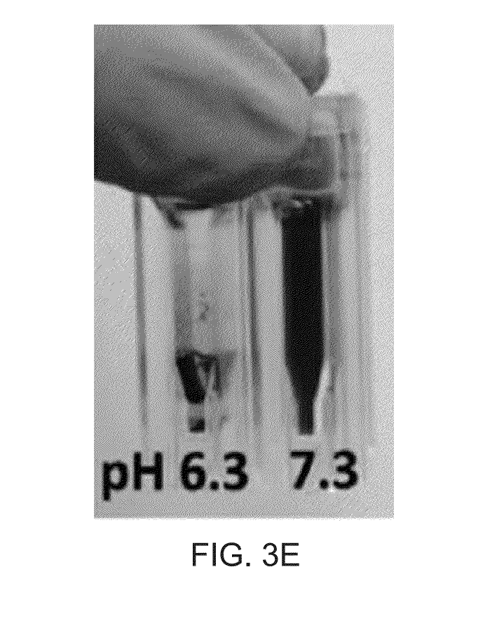

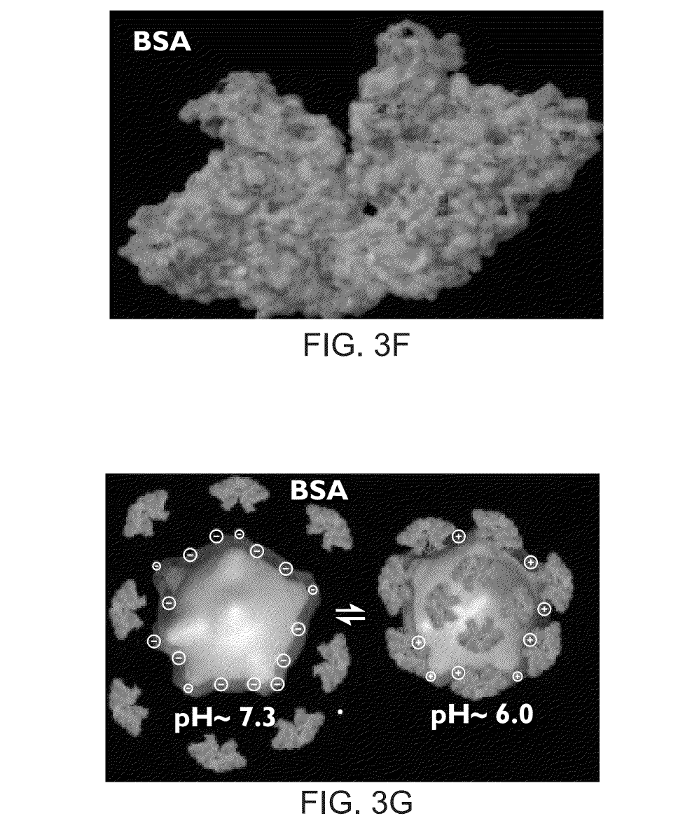

Nanoparticles intended for in vivo biomedical applications (e.g., imaging and therapy) possess high serum and plasma stability. In general, most of the naked metal nanoparticles experience the formation of a protein corona once they are exposed to physiological fluids (FIG. 3B). The protein corona is known to trigger immune response, eventually leading to clearance of the nanoparticles from blood circulation. Among other factors, the nature of the protein corona on nanoparticles is governed by the size, shape, surface charge and surface chemistry of the nanoparticles. Most nanoparticles previously developed require further processing to impart stealth character to these nanoparticles. However, such strategies have resulted only partial success making their translation to preclinical and clinical settings difficult. The chitosan aspect of the GRBs of the present disclosure can stabilize the GRB nanoparticle, as well as repel protein to reduce or prevent the formation of protein corona under certain conditions.

In various aspects, the GRBs can maintain stability when in circulation, but once inside a tumor can exhibit reduced stability and aggregate within the tumor. At physiological pH, GRBs can exhibit ξ-potential of about −30 mV with an effective hydrodynamic diameter of about 120 nm, whereas at pH about 6.5, the potential of the GRBs can be reversed to about +30 mV with a hydrodynamic diameter of about 120 nm (FIG. 3A). This pH dependent charge reversal behavior is similar to that exhibited by chitosan. For GRBs dispersed in 10% and 100% FBS at pH about 7.3 and 6.5, as depicted by Vis-NIR extinction spectra, even after 30 minutes of incubation at pH 7.3, the LSPR wavelength of GRBs does not exhibit any noticeable LSPR shift (FIG. 3C), demonstrating their excellent stability and the protein repellant activity of chitosan, under these pH conditions (FIG. 3C).

On the other hand, at pH 6.5, the extinction spectra of GRBs changes with the appearance of a broad extinction band at higher wavelength (about 800 nm), which may indicate aggregation of the GRBs in FBS as a result of protein corona around the GRBs (FIG. 3D). Visual inspection of the GRB solutions under these conditions can be used to confirm their stability at pH ranges such as from about 6.3 to about 7.3 (FIG. 3E).

The GRBs can aggregate as the pH is lowered, as indicated by FIG. 3B. At low pH, the positively charged GRBs tend to interact with negatively charged serum proteins e.g., bovine serum albumin (BSA) (net charge of BSA in complete medium is −20 mV) as shown in FIG. 3F. Whereas the GRBs exhibit stability at physiological pH (about 7.3), which provides a mechanism for GRBs to escape the immune system and to maximize the blood circulation time. At the same time, poor colloidal stability of GRBs at tumorigenic pH (about 6.3) allows the GRBs to preferentially accumulate at tumor sites.

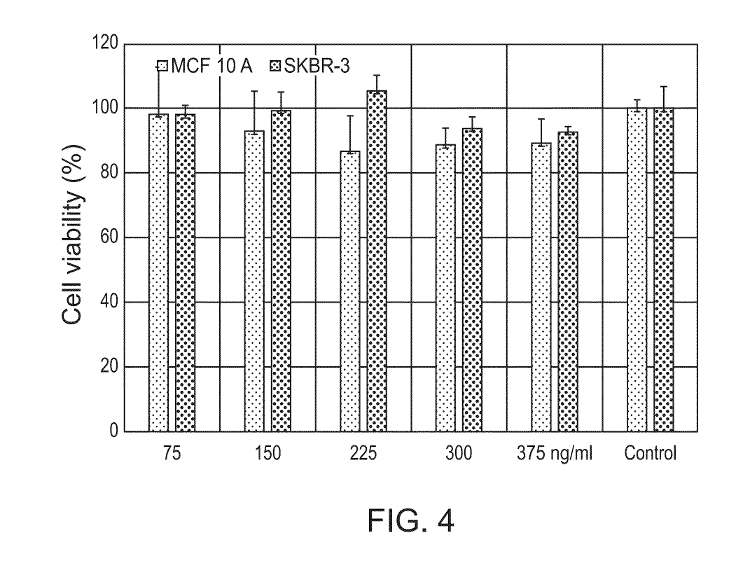

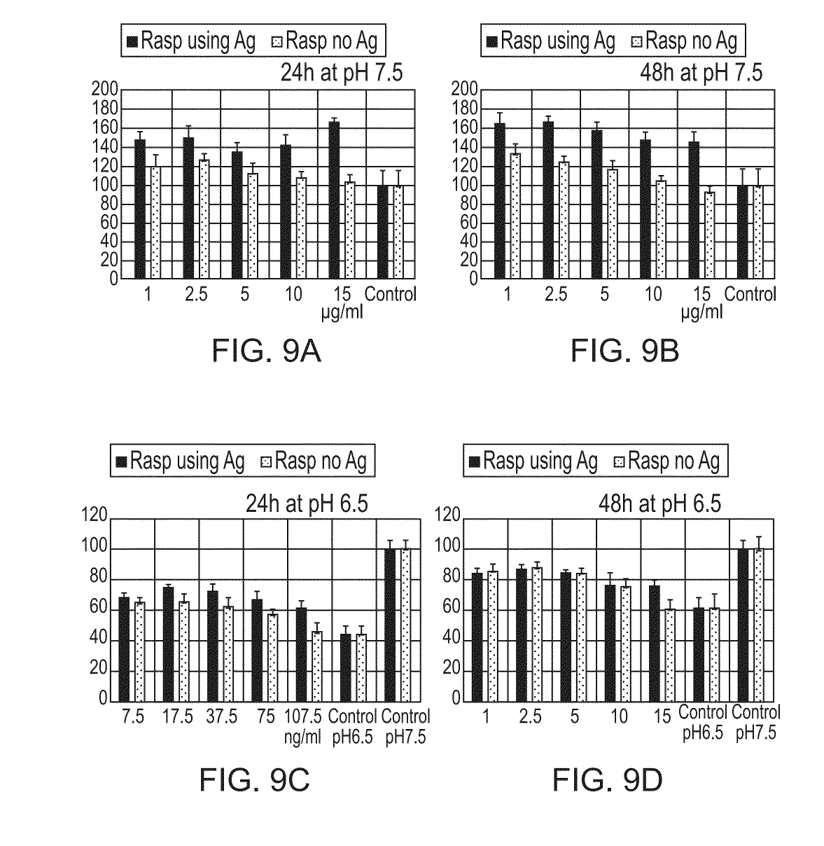

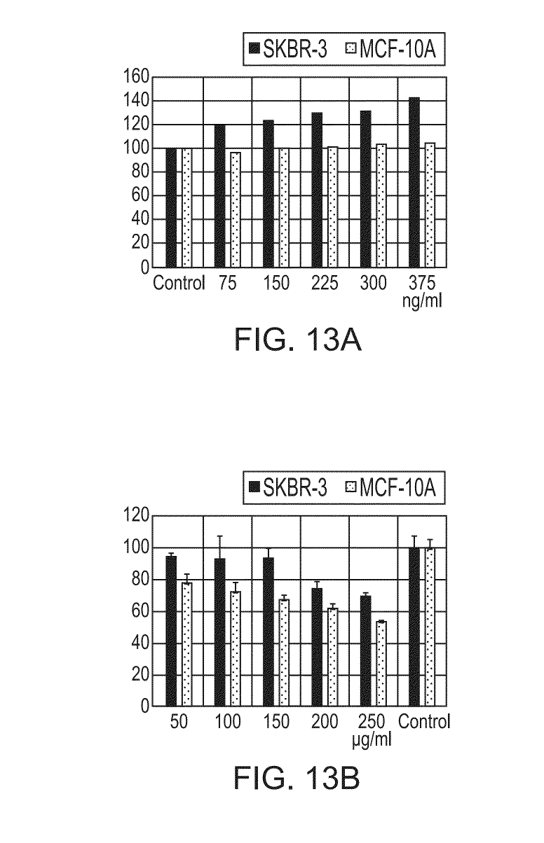

Cell lines show high cell viability (>90%) over a wide concentration range (25 to 375 ng/ml) of GRBs after 12, 24, 48 hours of incubation (FIGS. 4, 8, and 9). Trace amount of free chitosan in GRBs solution may lead to higher cell viability, while complete removal of free chitosan in the solution can reduce the cell viability. In an aspect, removal of free chitosan may reduce cell viability to about 90%. Without being limited to a particular theory, the reduction in cell viability without chitosan may be due to the oxidative stress caused by the metal nanoparticles (FIG. 4). In another aspect, low chitosan concentration (up to about 0.375 μg/ml) may promote the growth of cancer cells, but may not change healthy cell viability. For higher concentrations of chitosan (>50 μg/ml), the viability of both cancerous and healthy cells may be reduced (FIGS. 13A and 13B).

Cancer cells can preferentially uptake the GRBs over normal cells. Polysaccharides are known to internalize into several cancer types that overexpress folate receptors. Chitosan-coated GRBs exhibit significantly selective internalization into cancer cells. Without being bound by theory, breast cancer selectivity for GRBs of the present disclosure may be due to the over expressed glycoproteins. Furthermore, the change in pH within cancer tumors may contribute to the accumulation and aggregation of the GRBs within cancer tumors.

Cancer cells can then exhibit higher amounts of cell damage after incubation with GRBs and photothermal therapy. Without being bound by theory, the GRBs selectively accumulate in cancer cells, allowing for increased damage when a laser is directed at the cancer cells for photothermal therapy. The GRBs can have an localized surface plasmon resonance (LSPR) in the near infrared (NIR) therapeutic window of about 650 nm to about 900 nm. The target area can be irradiated with a laser with a wavelength ranging from about 550 nm to about 900 nm. In an aspect, a target area may be irradiated with a 808 nm diode laser with a power density of 370 mW/cm². Without being bound by theory, the GRBs that have accumulated within the cancer cells can heat and ablate the cancer cells while limiting damage to normal, healthy cells.

II. Synthesis of Nanoraspberries

In another aspect, the present disclosure is directed to a method of preparing a plurality of chitosan-coated gold nanoraspberries, the method comprising: forming a growth solution, wherein the growth solution is prepared by providing a chitosan solution; adding to the chitosan solution a solution comprising gold chloride (HAuCl₄); adding a solution comprising silver nitrate (AgNO₃) to the chitosan solution; adding ascorbic acid; and incubating the growth solution for a sufficient time to form the plurality of chitosan-coated gold nanoraspberries.

The GRBs do not require further procession or functionalization. Varying the concentration of the ingredients of the growth solution can affect the size and LSPR properties of the GRBs.

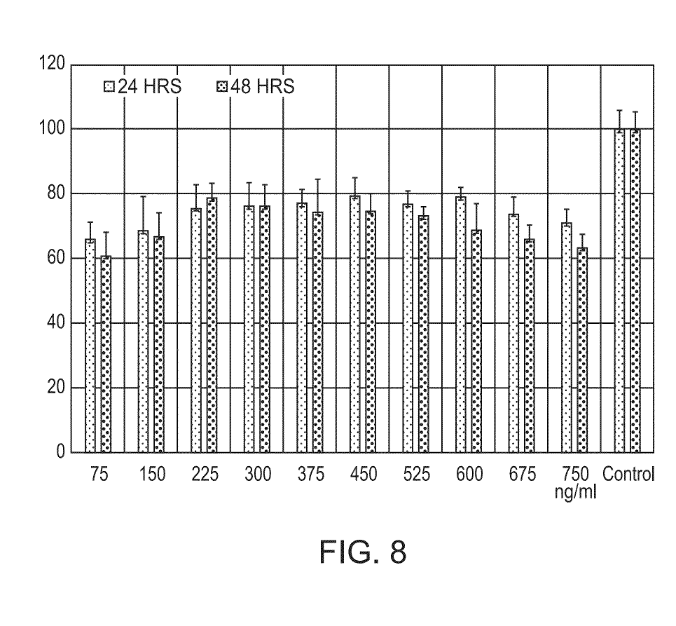

The chitosan solution comprises from about 0.5 mg/ml chitosan to about 10 mg/ml chitosan. GRBs can be synthesized using medium molecular weight (about 480,000 g/mol) chitosan (75-80% degree of deacetylation) as a soft template and capping agent. To aid in the solubility of chitosan in water, the pH of the aqueous solution is desirably maintained below 6.0 (pKa of chitosan is about 6.5) as illustrated in FIG. 8. The pH of the reaction as disclosed herein can be used to affect the rate, yield, and morphology of the GRBs. The amount of chitosan in the solution can range from about 0.5 mg/ml to about 10 mg/ml. The gold chloride has a concentration ranging from about 0.1 μmol/mg to about 0.5 μmol/mg. The ascorbic acid has a concentration ranging from about 0.01 μmol/mg to about 0.5 μmol/mg. In an exemplary GRB synthesis, 50 μL of HAuCl₄.4H₂O (4.86 mM), 2.5 μL of AgNO₃ (0.1 M), and 50 μL of ascorbic acid (0.1 M) are added to 10 ml of chitosan solution (1.25 mg/ml) at about pH 6. The reaction can be monitored by observing the color of the solution, which may gradually turn to pale/dark blue within 10 minutes depending on the concentration of chitosan. TEM images reveal the raspberry-like morphology of gold nanostructures obtained using this method (FIG. 1B).

The chitosan-coating has a thickness ranging from about 20 nm to about 30 nm.

The time can be from about 1 minute to about 24 hours. A particularly suitable time is from about 2 minutes to about 10 minutes.

One considerations in the design and synthesis of plasmonic nanostructures for in vivo biomedical applications is the ability to tune the LSPR of the nanostructures to NIR therapeutic window (650-900 nm), where the endogenous absorption coefficient of the tissue is nearly two orders magnitude lower compared to that in the visible part of EM spectrum. GRBs of the present disclosure offer facile tunability of the size and optical properties making them ideal for in vivo applications. In an aspect, GRBs may have an LSPR between about 650 nm and about 900 nm.

The size of GRBs can be varied by altering the concentration of chitosan in the growth solution. Thus, the amount of chitosan in the methods can be from about 0.5 mg/ml to about 10 mg/ml. Increasing the concentration of chitosan from 0.5 to 10 mg/ml can lead to a progressive decrease in the size of the GRBs and a concomitant blue shift in the LSPR band of GRBs (FIGS. 1C-1G and 10. The characteristic raspberry, or clustered, morphology of these GRBs can be preserved across different sizes. In addition to observing a blue shift in the LSPR band, GRB size can be monitored by analysis of electron microscopy images.

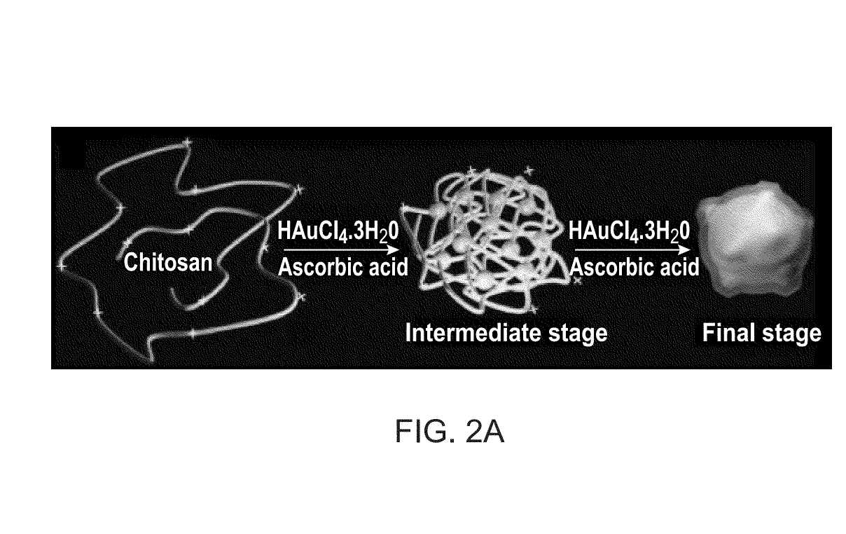

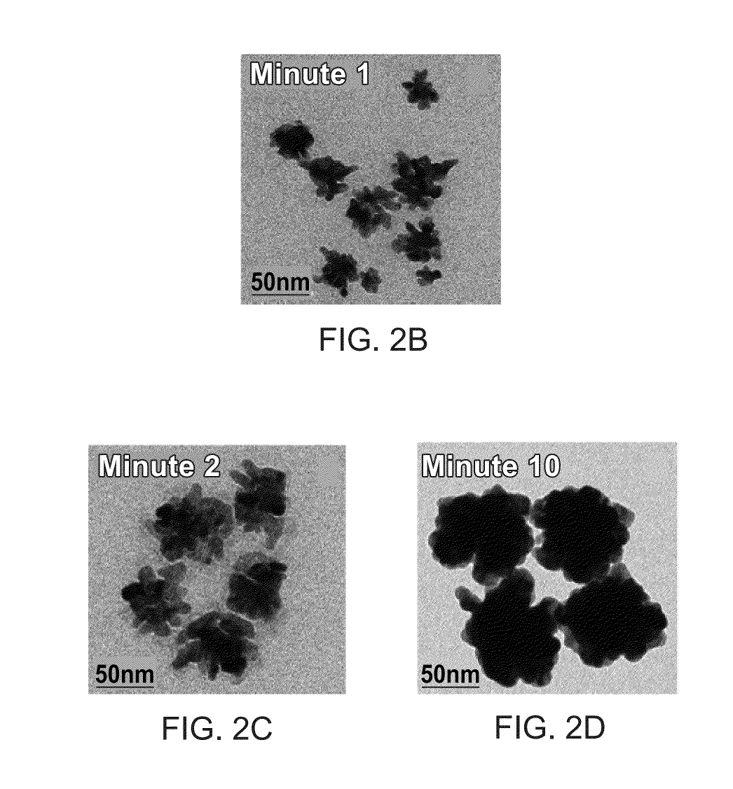

The method includes the addition of ascorbic acid (reducing agent) into the growth solution (FIGS. 2B-2D). Without being bound by theory, after the first minute of growth, there may be Au seeds that are not fully coalesced as evidenced by the tiny gaps within the branched nanostructures (FIGS. 2B and 2C). Subsequently, these disconnected seeds may continue to grow, leading to the formation of GRBs (FIGS. 2C and 2D). Chitosan is a relatively stiff polymer with a large persistence length (10-25 nm), causing the polymer chain conformation to resemble a highly open 3D scaffold. The protonated amine groups of chitosan that are known to have high affinity to Au may act as nucleation sites, forming tiny Au seeds along the chain, which upon subsequent growth may coalesce to form raspberry shaped Au nanostructures.

In another aspect, the present disclosure is directed to a method of photothermal cancer treatment in a subject having or suspected of having a cancer tumor, the method comprising: administering a plurality of chitosan-coated gold nanoraspberries to the subject; incubating the subject for a sufficient period of time to allow for internalization of the chitosan-coated gold nanoraspberries by cells of the cancer tumor; and exposing the cancer tumor to laser irradiation.

Particularly suitable cancers are tumor cancers. A particularly suitable tumor cancer is breast cancer. A particularly suitable breast cancer is an epithelial cell breast cancer.

Suitable laser irradiation has a wavelength ranging from about 550 nm to about 900 nm.

The period of time to allow for internalization of the chitosan-coated gold nanoraspberries by cells of the cancer tumor ranges from about 12 hours to about 48 hours.

The concentration of chitosan-coated gold nanoraspberries administered can range from about 25 ng/ml to about 150 ng/ml.

### EXAMPLES

### Example 1

### Materials

All materials were used as received without any further purification. Gold chloride (HAuCl₄.4H₂O), ascorbic acid, chitosan (medium molecular weight), 1-ethyl-3-(3-dimethylaminopropyl) carbodiimide (EDC), N-hydroxysuccinimide (NHS), fluorescein isothiocyanate (FITC), Dimethylthiazol-2-yl)-2,5-diphenyltetrazolium bromide (MTT) and pencillin-steptomycin were purchased from Sigma-Aldrich (St. Louis, Mo., USA). Hydrochloric acid (HCl) was obtained from EMD (Gibbstown, N.J). Live/Dead Viability kit (Ethidium homodimer-1 and Calcein AM) and Trypsin-EDTA (0.25% 1×) were purchased from Life Technologies Corp. McCoy's 5A medium, MEBM medium, MCF-10A cells, and SKBR3 cells were purchased from ATCC. MEGM bullet kit to mix with MEBM medium was purchased from Lonza (Kit Catalog No. CC-3150). The formvar/carbon coated copper TEM grids were acquired from Ted Pella (Redding, Calif., USA). Nanopure water (>18.0 Mω-cm) was used for all experiments.

### Example 2

### Synthesis of Chitosan Protected Gold Nanoraspberries

The chitosan solution used in the synthesis of gold nanoraspberries was made by dissolving 50 mg of medium molecular weight chitosan in 3 mL of water at pH 1.4. Once the chitosan was completely dissolved after vigorous sonication and vortexing, an additional 7 mL of water was added to the concentrated chitosan solution, resulting in a final concentration of 5 mg/mL. The pH of the chitosan solution at this stage was about 6.0. 200 μL of the chitosan solution (5 mg/mL) was then added to 800 μL it of water and the solution was homogenized by vortexing the solution. To this chitosan solution (1 mg/ml), 100 μL of gold chloride (4.86 mM) solution was added. The resultant solution was homogenized thoroughly to ensure the uniform solution. 50 μL of ascorbic acid (0.1 M) was added to the above reaction mixture under vigorous stirring (1200 rpm) for 30 seconds. The solution was left undisturbed for overnight to form gold nanoraspberries.

To understand the pH-dependent surface state of chitosan-coated GRBs, their size and zeta-potential were measured at both physiological (about pH 7.5) and tumorigenic (about pH 6.5) conditions (FIGS. 10A-10D). At physiological pH, GRBs exhibit a ζ-potential of −30 mV with an effective hydrodynamic diameter of 120 nm, whereas at about pH 6.5, the -potential of the nanostructures was completely reversed to +30 mV with a hydrodynamic diameter of 120 nm (FIG. 3A). This pH dependent charge reversal behavior is similar to that exhibited by chitosan, which further confirmed the presence of chitosan on the GRBs. The serum stability of GRBs dispersed in 10% and 100% FBS was observed at about pH 7.3 and 6.5. As depicted by vis-NIR extinction spectra, even after 30 minutes of incubation at pH 7.3, the LSPR wavelength of GRBs did not exhibit any noticeable LSPR shift (FIG. 3C), which indicated their excellent stability and that chitosan, under these pH conditions, effectively acts as a protein repellant (FIG. 3C). On the other hand, at pH 6.5, the extinction spectra of GRBs showed a dramatic change with the appearance of a broad extinction band at higher wavelengths (about 800 nm), which indicated aggregation of the nanoparticles in FBS as a result of protein corona around the nanoparticles (FIG. 3D). Visual inspection of the nanoparticle solutions at these conditions confirmed their stability at about pH 7.3 and lack of thereof at about 6.3 (FIG. 3E).

To further understand protein corona formation and colloidal stability of GRBs, the hydrodynamic diameter of these nanoparticles was monitored using dynamic light scattering (DLS) for the first 30 min after adding 10% FBS to the nanoparticle solution. At pH 7.3, the hydrodynamic diameter of GRBs (about 110 nm) remained virtually unchanged even 30 minutes after adding 10% FBS. On the other hand, at pH 6.0, the hydrodynamic diameter of the GRBs monotonically increased up to 3 μm within 30 minutes, indicating the strong aggregation of the nanoparticles in solution (FIG. 3B). At low pH, the positively charged GRBs tend to interact with negatively charged serum proteins e.g., bovine serum albumin (BSA) (net charge of BSA in complete medium is −20 mV) as shown in FIGS. 3F and 3G. Serum stability studies indicated that the GRBs exhibit excellent stability at physiological pH (about 7.3), which allows the GRBs to escape the immune system and to maximize their blood circulation time. At the same time, poor colloidal stability of GRBs at tumorigenic pH (about 6.3) causes them to preferentially accumulate at the tumor site.

### Example 3

### FITC-Conjugation

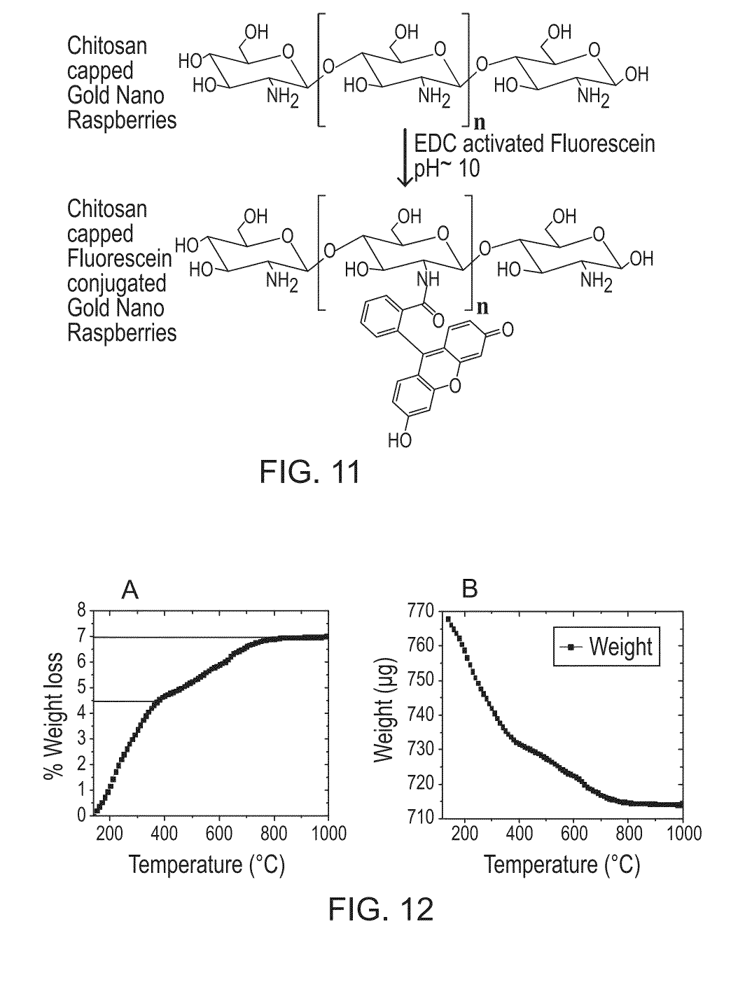

1 mL of 10 μmol fluorescein sodium salt (FITC) solution in water was activated with 10 μmol 1-Ethyl-3-(3-dimethylaminopropyl) carbodiimide (EDC). Then 50% of free amines on chitosan (2 μmol of monomer concentration) were modified using 15 μmol of N-hydroxysuccinimide and 10 μmol of activated FITC. Then the pH of the reaction was slowly adjusted to about 6.5 and the reaction was left overnight. Subsequently, the pH of the reaction was adjusted to basic (about 9) to precipitate chitosan-GRBs and washed 5 times to completely remove the free FITC. Then FITC-GRBs conjugation was confirmed by UV/Vis and FT-IR (FIG. 11).

### Example 4

### Cell Culture

Human epithelial breast cells (MCF-10A) and breast cancer cells (SKBR3) were purchased from ATCC (Manassas, Va.) and sub-cultured. MCF-10A cells were sub-cultured in base medium (MEBM) along with the additives obtained from Lonza/Clonetics Corporation (MEGM, Kit Catalog No. CC-3150). SKBR-3 cells were cultured in McCoy's 5A medium with 10% fetal bovine serum (FBS) and antibiotics (100 μg/ml penicillin and 100 μg/ml streptomycin) (Sigma, St. Louis, Mo.). Both the cell lines were grown in water jacket incubator at 3TC with 5% CO₂-humidified atmosphere in 25 cm² tissue culture flasks. Once the cells reached to 90% confluence, they were washed with phosphate buffered saline (PBS) and detached with 1 mL of 0.25% trypsin-EDTA solution (Sigma). Cells were dispersed in 10 ml complete medium with 10% FBS and centrifuged. Cells were counted in a disposable hemocytometer and plated at a density of 5×10⁵ and 4×10⁴ cells in flat bottom 24 well and 96 well plates (Corning Life Sciences), respectively. To co-culture, equal number (2×10⁵) of SKBR-3 and MCF-10A cells were plated in 24 well plates using MEBM as medium. MEBM did not cause any damage to SKBR-3 cells, indicating that MEBM can be used to culture both cell lines without significant cell damage.

### Example 5

### In Vitro Photothermal Studies

Photothermal studies of MCF-10A, SKBR-3, and co-culture cells with and without gold nanoraspberries were conducted using 808 nm diode laser with a power density of 370 mW/cm². At this power density, no cell damage was observed to either of the cell types, indicating that the laser power used was safe. To distinguish live and dead cells following the photothermal therapy, the cells were incubated with ethidium homobromide-1 and calcein AM dyes to produce green and red emission from live and dead cells, respectively.

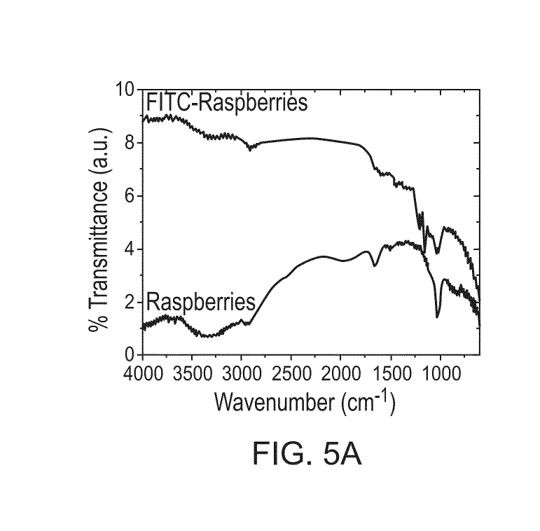

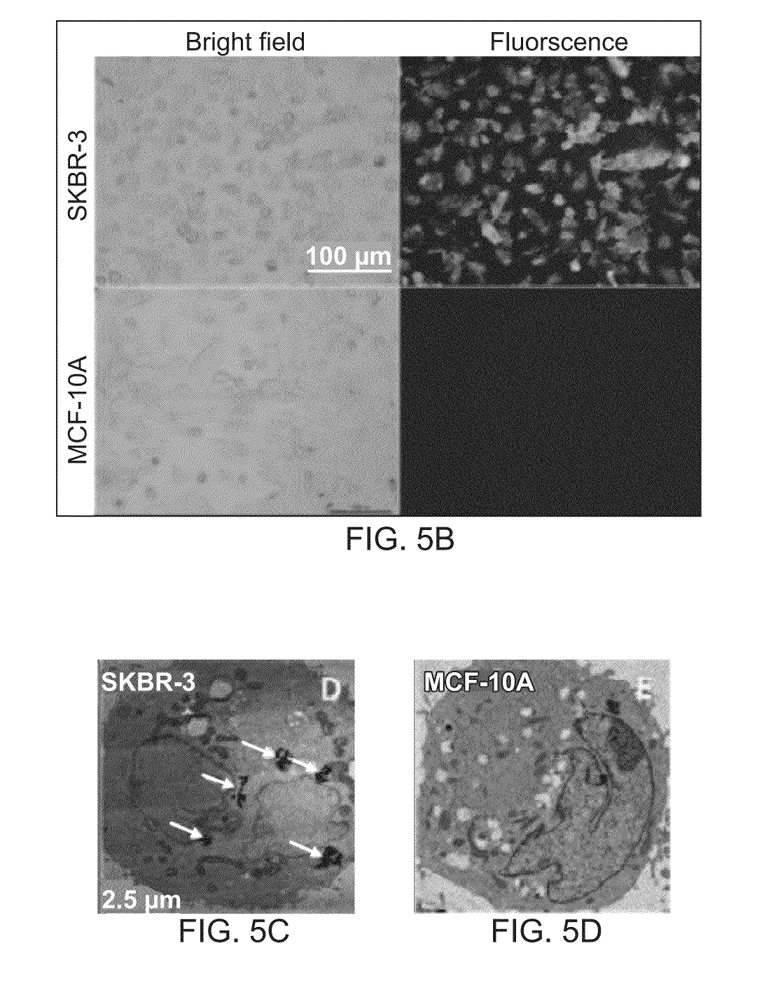

To confirm the cancer selective internalization, the internalization of GRBs was explored in both MCF-10 A (negative control) and SKBR-3 (positive control) cells. To study the cancer selectivity of GRBs using fluorescence microscopy, fluorescein isothiocyanate (FITC) was conjugated to the free amine groups of chitosan using carbodimide chemistry. The successful conjugation resulted in an absorption peak corresponding to FITC at 455 nm in Vis-NIR extinction spectra of GRBs. Fourier transform infrared (FT-IR) spectra of FITC-GRBs indicated the difference in relative intensities of primary and secondary amine peaks at 3300 cm⁻¹ and 2900 cm⁻¹ compared to unmodified chitosan, which is a direct evidence of successful conjugation of FITC to chitosan (FIG. 5A).

To monitor the internalization ability of GRBs, MCF-10A and SKBR-3 cells were incubated with FITC-conjugated GRBs for 6 hours at 37° C. in humidified atmosphere with 5% bone dry CO₂. After 6 hours of incubation, cells were fixed using 4% formaldehyde and permeabilized in 1% TRITON X-100 for 15 minutes and washed thoroughly using Dulbecco's phosphate buffered saline (DPBS). The fixed cells on cover slips were analyzed using epifluorescence microscopy (FIG. 5B). The high uptake of GRBs by SKBR-3 cells was evidenced by bright green fluorescence from SKBR-3 cells. On the other hand, no perceivable green fluorescence was observed in MCF-10A, which confirmed the selective internalization of GRBs into SKBR-3. To further confirm the localization of GRBs inside the cell, TEM imaging of ultrathin sections (60-90 nm) of SKBR-3 and MCF-10A cells following their incubation with GRBs was performed. TEM imaging revealed numerous nanoparticles accumulated within the SKBR-3 cells (FIG. 5C), whereas the sections from MCF-10A cells did not reveal any particles inside the cells (FIG. 5D). Taken together, cell viability and internalization studies indicate that chitosan-coated GRBs exhibit selective internalization into SKBR-3 cells.

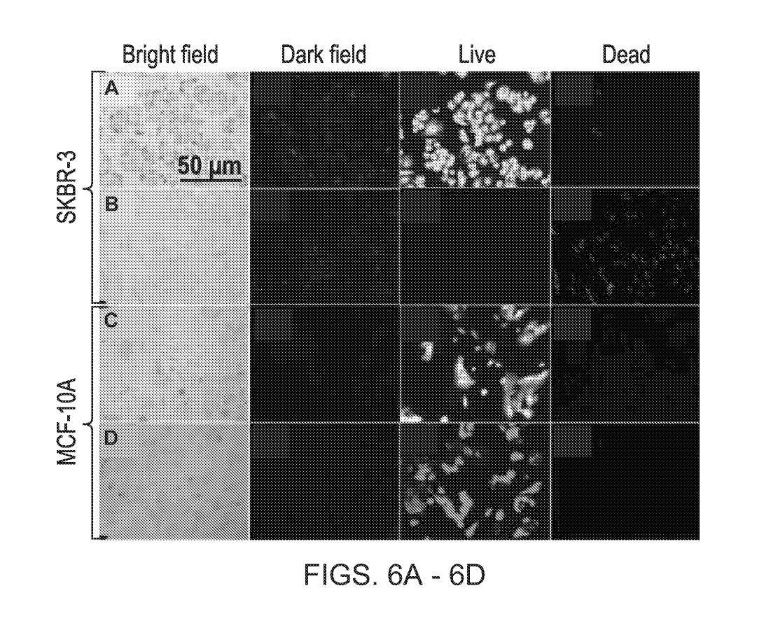

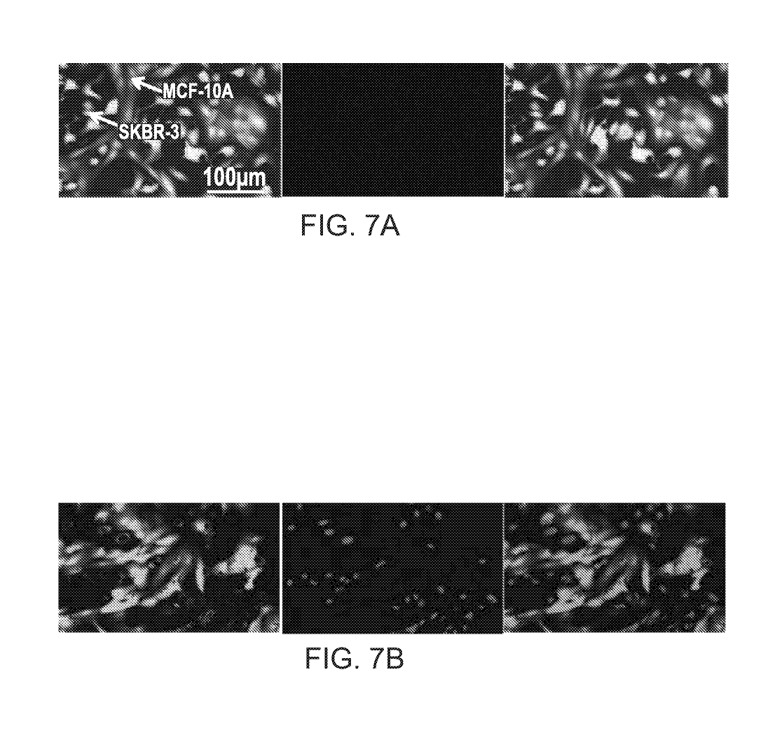

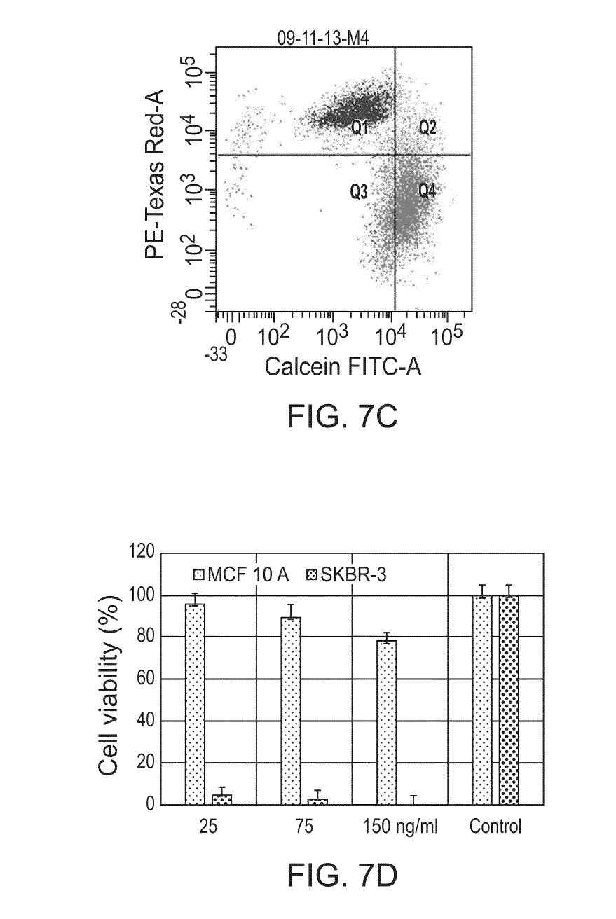

Once the selective internalization of GRBs was confirmed, in vitro photothermal studies were performed on MCF-10A, SKBR-3 and co-cultures of MCF-10A and SKBR-3 (FIGS. 6 and 7). Photothermal studies with GRBs as contrast agents were performed on SKBR-3 and MCF-10A cell lines using a commercially available live/dead viability kit (green color for live and red for dead) as shown in FIGS. 6 and 7. The cells in rows of FIGS. 6A and 6B correspond to SKBR-3. The cells in rows of FIGS. 6C and 6D correspond to MCF-10A. Both the cell lines were incubated with 10 ng/ml of GRBs for 12 hours before laser exposure (808 nm). Images in rows of FIGS. 6A and 6C correspond to cells that were not treated with laser. Images in rows of FIGS. 6B and 6D correspond to cells that were irradiated with laser at a power density of 320 mW/cm² for 3 minutes. The fluorescence images were collected after exposing the cells to live/dead staining solution for 30 minutes. The control cells i.e., cells that were incubated with GRBs but not exposed to laser, showed bright green fluorescence, which corresponds to live cells and indicated that the GRBs alone did not result in any significant cell death (FIGS. 6A and 6C). Laser irradiation of SKBR-3 cells that were incubated with GRBs resulted in significant cell death (FIG. 6D). On the other hand, laser irradiation of MCF-10A cells incubated with GRBs did not result in significant cell death as evidenced by bright green fluorescence and absence of red fluorescence (FIG. 6D). These observations agreed with the GRBs internalization studies, which demonstrated the large uptake of GRBs by SKBR-3 cells but absence of uptake by MCF-10A.

To further demonstrate the selective photothermal cancer therapy in vitro, selective cell killing experiments were conducted on co-culture of SKBR-3 and MCF-10A cells that were incubated with GRBs (FIGS. 7A and 7B). Due to the preferential uptake of GRBs into cancer cells, SKBR-3 cells were completely damaged after photothermal therapy as indicated by observance of red fluorescence. The green fluorescence in the same image indicated live MCF-10A cells, demonstrating that the photothermal therapy was highly selective to breast cancer cells. Flow cytometry was used to count live and dead cells after photothermal therapy in co-cultured cells. As depicted in FIG. 7C, about 50% of the cells were stained with red and about 50% of the cells were stained with green, further confirming that about half of the co-cultured cells were dead due to the targeted photothermal therapy. This result was consistent with the live/dead fluorescence imaging of co-cultured cells after photothermal therapy. Cell viability after photothermal therapy was also estimated using MTT studies (FIG. 7D). Even at very low concentration of GRBs, SKBR-3 cells were completely dead immediately after laser exposure whereas MCF-10A cells showed about 95% viability. Taken together, photothermal studies performed on individual cell cultures and co-cultures demonstrated the cancer specificity of GRBs.

### Example 6

### Characterization

TEM images were obtained using FEI sprint Lab6 with an accelerating voltage of 120 kV. UV-vis-NIR extinction spectra were collected using a Shimadzu 1800 spectrophotometer. Hydrodynamic area and zeta potential of GRBs were measured using Dynamic Light Scattering (Malvern Zetasizer Nano S/ZS). Fourier Transform Infrared-Red spectra of GRBs and FITC-GRBs powder were measured using smart performer (attenuated total reflectance (ATR) accessory) in Nicolette Nexus 470. Thermogravimetric analysis of GRBs was performed by Q5000 IR thermogravimetric analyzer (TA instruments).

To confirm the presence of a chitosan layer and estimate the thickness of the chitosan layer on GRBs, 2% uranyl acetate was used to negatively stain the TEM grids. TEM imaging revealed a chitosan polymer layer having a thickness of about 20 nm to about 30 nm on GRBs (FIG. 1H). The thickness of the chitosan layer on GRBs obtained from TEM analysis was consistent with the hydrodynamic diameter of GRBs measured using dynamic light scattering (FIG. 10C). To further estimate the amount of chitosan on GRBs, thermogravimetric analysis (TGA) was performed on GRBs powder. Thermogravimetric analysis revealed about 2.5% organic (chitosan) and about 97.5% of inorganic (gold) content in the GRB sample (FIGS. 12A and 12B).

To analyze the GRBs growth mechanism, TEM samples were prepared and analyzed at three different time points (1, 2 and 10 minutes) after the addition of ascorbic acid (reducing agent) into the growth solution (FIGS. 2B-2D). TEM images obtained after first minute of growth revealed Au seeds that were not fully coalesced as evidenced by the tiny gaps within the branched nanostructures (FIGS. 2B and 2C). Subsequently, the disconnected seeds continue to grow, leading to the formation of GRBs as seen in TEM images at t=2 and 10 minutes (FIGS. 2C and 2D). Chitosan is a relatively stiff polymer with a large persistence length (10-25 nm), causing the polymer chain conformation to resemble a highly open 3D scaffold. The protonated amine groups of chitosan that are known to have high affinity to Au possibly act as nucleation sites, forming tiny Au seeds along the chain, which upon subsequent growth coalesce to form raspberry shaped Au nanostructures (FIG. 2A).

MTT (3-(4,5-dimethylthiazol-2-yl)-2,5-diphenyltetrazolium bromide) assay was performed to evaluate the cytotoxicity of GRBs (75 to 375 ng/ml) in both MCF-10A (epithelial breast cells) and SKBR-3 (epithelial breast cancer cells) cells (FIG. 4). Both the cell lines showed high cell viability (>90%) over a wide GRBs concentration range (25 to 375 ng/ml) after 12, 24, 48 hours of incubation with GRBs (FIGS. 4, 8, and 9). No significant drop in cell viability was observed for both SKBR-3 and MCF-10A cells even at very high concentration of GRBs (375 ng/ml), indicating the biocompatible nature these nanoparticles. The trace amount of free chitosan in GRBs solution appears to lead to higher cell viability of SKBR-3 cells. However, after complete removal of free chitosan in the solution, the cell viability dropped to 90%, which may be due to the oxidative stress caused by the metal nanoparticles (FIG. 4). To better understand the effect of free chitosan on the cell viability, MTT studies were conducted using different concentration of chitosan (0.075 to 250 μg/ml). Low chitosan concentration (up to 0.375 μg/ml) promoted the growth of SKBR-3 cells, but no major change was observed in MCF-10A cell viability. For higher concentrations of chitosan (>50 μg/ml), the viability of both SKBR-3 and MCF-10A cells significantly reduced (FIGS. 13A and 13B). Between about 0.075 μg/ml and about 37.5 μg/ml, the viability of SKBR-3 cell was higher than the control cells. No significant difference was noted in the case of MCF-10A cells. Once the concentration levels increased to 50 μg/ml, the cell viability of both cell lines dropped.

The examples described herein are included to demonstrate preferred embodiments of the invention. It should be appreciated by those of skill in the art that the techniques disclosed in the examples included herein represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

What is claimed is:

1. A composition comprising:
a plurality of gold nanoparticles; and a chitosan-coating surrounding the plurality of gold nanoparticles, wherein the composition has a raspberry-like morphology.

2. The composition of claim 1, wherein the composition has a diameter ranging from about 100 nm to about 150 nm.

3. The composition of claim 1, wherein the composition has a localized surface plasmon resonance peak ranging from about 650 nm to about 900 nm.

4. The composition of claim 1, wherein the chitosan coating has a thickness ranging from about 20 nm to about 30 nm.

5. The composition of claim 1, wherein the composition comprises about 1% to about 10% chitosan content as measured by thermogravimetric analysis.

6. The composition of claim 1, wherein the composition comprises about 90% to about 99% gold content as measured by thermogravimetric analysis.

7. A method of preparing a plurality of chitosan-coated gold nanoraspberries, the method comprising: forming a growth solution, wherein the growth solution is prepared by providing a chitosan solution; adding to the chitosan solution a solution comprising gold chloride (HAuCl₄); adding a solution comprising silver nitrate (AgNO₃) to the chitosan solution; adding ascorbic acid; and incubating the growth solution for a sufficient time to form the plurality of chitosan-coated gold nanoraspberries.

8. The method of claim 7, wherein the chitosan solution comprises from about 0.5 mg/ml chitosan to about 10 mg/ml chitosan.

9. The method of claim 7, wherein the chitosan has a molecular weight of about 480,000 g/mol.

10. The method of claim 7, wherein the chitosan solution has a pH of about 6.0.

11. The method of claim 7, wherein the gold chloride has a concentration ranging from about 0.1 μmol/mg to about 0.5 μmol/mg.

12. The method of claim 7, wherein the ascorbic acid has a concentration ranging from about 0.01 μmol/mg to about 0.5 μmol/mg.

13. The method of claim 7, wherein the chitosan-coating has a thickness ranging from about 20 nm to about 30 nm.

14. The method of claim 7, wherein the time is from about 1 minute to about 24 hours.

15. The method of claim 7, wherein the time is from about 2 minutes to about 10 minutes.

16. A method of photothermal cancer treatment in a subject having or suspected of having a cancer tumor, the method comprising: administering a plurality of chitosan-coated gold nanoraspberries to the subject; incubating the subject for a sufficient period of time to allow for internalization of the chitosan-coated gold nanoraspberries by cells of the cancer tumor; and exposing the cancer tumor to laser irradiation.

17. The method of claim 16, wherein the cancer is breast cancer.

18. The method of claim 16, wherein the laser irradiation has a wavelength ranging from about 550 nm to about 900 nm.

19. The method of claim 16, wherein the period of time ranges from about 12 hours to about 48 hours.

20. The method of claim 16, wherein the concentration of chitosan-coated gold nanoraspberries administered ranges from about 25 ng/ml to about 150 ng/ml.
